(19) United States
(12) Patent Application Publication
(10) Pub. No.: US 2025/0332426 A1
(43) Pub. Date: Oct. 30, 2025

(54) IMPLANTABLE MEDICAL DEVICE WITH BIOCOMPATIBLE ELECTRICAL INSULATOR

(71) Applicant: Medtronic, Inc., Minneapolis, MN (US)

(72) Inventors: Emma L. Yurs, Minneapolis, MN (US); Jake H. Kanack, Columbia Heights, MN (US); Robert A. Munoz, Andover, MN (US); Everett A. Kroll, Woodbury, MN (US); Silvia Tinelli, Coralville, IA (US); Thomas W. Hovind, Andover, MN (US)

(21) Appl. No.: 19/188,527

(22) Filed: Apr. 24, 2025

Related U.S. Application Data
(60) Provisional application No. 63/639,374, filed on Apr. 26, 2024.

(51) Int. Cl. A61N 1/375; B23K 26/36

(52) U.S. Cl. CPC A61N 1/3756; B23K 26/36

(57) ABSTRACT

An example method of manufacturing an implantable medical device includes disposing a biocompatible electrical insulator on an outer surface of a housing of the implantable medical device and to cover an outer surface of an electrode that is positioned on the outer surface of the housing, ablating a portion of the biocompatible electrical insulator, and removing the biocompatible electrical insulator to expose the outer surface of the electrode.

### RELATED APPLICATIONS

This application claims the benefit of U.S. Provisional Application Ser. No. 63/639,374, filed Apr. 26, 2024, the entire contents of each of which are incorporated herein by reference.

### TECHNICAL FIELD

The disclosure relates to implantable medical devices.

### BACKGROUND

Various implantable medical devices (IMDs) have been clinically implanted or proposed for therapeutically treating or monitoring one or more conditions of a patient. Such devices may be adapted to monitor or treat conditions or functions relating to heart, muscle, nerve, brain, stomach, endocrine organs or other organs and their related functions. Advances in design and manufacture of miniaturized electronic and sensing devices have enabled development of implantable devices capable of therapeutic as well as diagnostic functions such as pacemakers, cardioverters, defibrillators, biochemical sensors, implantable loop recorders, and pressure sensors, among others. Such devices may be associated with leads that position electrodes or sensors at a desired location or may be leadless with electrodes integrated into the device housing. These devices may have the ability to wirelessly transmit data either to another device implanted in the patient or to another instrument located externally of the patient, or both.

Although implantation of some devices requires a surgical procedure (e.g., pacemakers, defibrillators, etc.), other devices may be small enough to be delivered and placed at an intended implant location in a relatively noninvasive manner, such as by a percutaneous delivery catheter, transvenously, or using a subcutaneous delivery tool. As one example, subcutaneously implantable monitors have been proposed and used to monitor heart rate and rhythm, as well as other physiological parameters, such as patient posture and activity level. Such direct in vivo measurement of physiological parameters may provide significant information for clinicians to facilitate diagnostic and therapeutic decisions.

### SUMMARY

The disclosure describes implantable medical devices (IMDs) including a biocompatible electrical insulator to electrically isolate an electrode (e.g., a sensing electrode) of the IMD, and associated techniques for manufacturing IMDs including a biocompatible electrical insulator. An IMD includes a housing and an electrode positioned on an outer surface of the housing. The IMD also includes a biocompatible electrical insulator disposed on the outer surface of the housing and electrode, with a portion of the biocompatible electrical insulator removed, via precision removal, from the outer surface of the electrode.

In one example, this disclosure describes a method of manufacturing an implantable medical device, the method including: disposing a biocompatible electrical insulator on an outer surface of a housing of the implantable medical device and to cover an outer surface of an electrode that is positioned on the outer surface of the housing; ablating a portion of the biocompatible electrical insulator; and removing the biocompatible electrical insulator to expose the outer surface of the electrode.

In another example, this disclosure describes implantable medical device including: a housing configured to house processing circuitry, wherein the processing circuitry is configured to control functioning of the implantable medical device, wherein the housing includes: an electrically conductive portion defining a cavity configured to receive the processing circuitry; and a dielectric cover configured to cover the cavity and enclose the processing circuitry within the cavity; an electrode positioned on an outer surface of the dielectric cover and opposite the processing circuitry, wherein the processing circuitry is configured to monitor a physiological parameter of a patient via the electrode; and a biocompatible electrical insulator disposed on an outer surface of at least one of the electrically conductive portion or the dielectric cover, wherein a first portion of the biocompatible electrical insulator is ablated, and wherein a second portion of the biocompatible electrical insulator is removed to expose an outer surface of the electrode.

In another example, this disclosure describes a method including: disposing a biocompatible electrical insulator on an outer surface of a housing of an implantable medical device and to cover an outer surface of an electrode, wherein the housing includes: an electrically conductive portion defining a cavity configured to receive processing circuitry that is configured to control functioning of the implantable medical device; and a dielectric cover configured to cover the cavity and enclose the processing circuitry within the cavity, wherein the electrode is positioned on a portion of the outer surface of the dielectric cover; ablating a portion of the biocompatible electrical insulator to expose an outer surface of the electrode; and ablating the surface of the electrode to form a surface texture that increases a surface area of the surface of the electrode concurrently with ablating the portion of the biocompatible electrical insulator.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below. Other features, objects, and advantages will be apparent from the description, drawings, and claims.

### BRIEF DESCRIPTION OF THE FIGURES

The details of one or more examples of this disclosure are set forth in the accompanying drawings and the description below. Other features, objects, and advantages of this disclosure will be apparent from the description, drawings, and claims.

FIG. 1 is a conceptual drawing illustrating an example medical device system in conjunction with a patient, according to various examples described in this disclosure.

FIG. 2A is a conceptual cross-sectional side-view diagram illustrating an example configuration of the implantable medical device (IMD) and biocompatible electrical insulator of the medical system of FIG. 1, according to various examples described in this disclosure.

FIG. 2B is a conceptual perspective view diagram illustrating the example configuration of the implantable medical device (IMD) and biocompatible electrical insulator of FIG. 2A, according to various examples described in this disclosure.

FIG. 3 is a functional block diagram illustrating an example configuration of the implantable medical device (IMD) of the medical system of FIG. 1, according to various examples described in this disclosure.

FIG. 4A is a conceptual cross-sectional side-view illustrating a configuration of a portion of IMD.

FIG. 4B is a conceptual cross-sectional side-view illustrating another configuration of a portion of IMD.

FIG. 4C is a conceptual cross-sectional side-view illustrating another configuration of a portion of IMD.

FIG. 5 is a flow diagram of an example method of manufacturing an implantable medical device including a biocompatible electrical insulator, according to various examples described in this disclosure.

FIG. 6A is a conceptual cross-sectional side-view illustrating a configuration of a portion of IMD, according to various examples described in this disclosure.

FIG. 6B is a conceptual cross-sectional side-view illustrating another configuration of a portion of IMD, according to various examples described in this disclosure.

FIG. 6C is a conceptual cross-sectional side-view illustrating another configuration of a portion of IMD, according to various examples described in this disclosure.

FIG. 6D is a conceptual cross-sectional side-view illustrating another configuration of a portion of IMD, according to various examples described in this disclosure.

FIG. 7A is a conceptual cross-sectional side-view illustrating a configuration of a portion of IMD, according to various examples described in this disclosure.

FIG. 7B is a conceptual cross-sectional side-view illustrating another configuration of a portion of IMD, according to various examples described in this disclosure.

FIG. 7C is a conceptual cross-sectional side-view illustrating another configuration of a portion of IMD, according to various examples described in this disclosure.

FIG. 7D is a conceptual cross-sectional side-view illustrating another configuration of a portion of IMD, according to various examples described in this disclosure.

FIG. 8 is a scanning electron microscopy (SEM) image of an example TiN surface structure, according to various examples described in this disclosure.

In the figures, use of a same reference number or a same reference number with a letter extension may be used to indicate a same or corresponding device or element when used in a same drawing or in different drawings. In addition, unless otherwise indicated, devices and/or other objects such as a patient, an implantable medical device, or an electrical device such as an electrical coil, are not necessarily illustrated to scale relative to each other and/or relative to an actual example of the item being illustrated. In particular, various drawings provided with this disclosure illustrate a “patient” represented by a human-shaped outline and are not to be considered drawn to scale relative to an actual human patient or with respect to other objects illustrated in the same figure unless otherwise specifically indicated in the figure for example by dimensional indicators, or for example as otherwise described in the text of the disclosure.

### DETAILED DESCRIPTION

A variety of types of medical devices sense physiological signals or parameters of a patient, such as cardiac electrograms (EGMs) and/or other. Some medical devices that sense patient signals or parameters are non-invasive, e.g., using a plurality of electrodes (e.g., sensing electrodes) placed in contact with external portions of the patient, such as at various locations on the skin of the patient to sense patient signals or parameters, e.g., cardiac EGMs. The electrodes used to monitor the patient signals or parameters in these non-invasive processes may be attached to the patient using an adhesive, strap, belt, or vest, as examples, and electrically coupled to a monitoring device, such as an electrocardiogramalter monitor, or other electronic device. The electrodes are configured to sense electrical signals associated with the electrical activity of tissue of the patient, e.g., the heart or other cardiac tissue of the patient, and to provide these sensed electrical signals to the electronic device for further processing and/or display of the electrical signals. The non-invasive devices and methods may be utilized on a temporary basis, for example to monitor a patient during a clinical visit, such as during a doctor's appointment, or for example for a predetermined period of time, for example for one day (twenty-four hours), or for a period of several days.

External devices that may be used to non-invasively sense and monitor patient signals or parameters include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities and may periodically transmit collected data to a network service, such as the Medtronic Carelink™ Network.

Some implantable medical devices (IMDs) also sense and monitor patient signals or parameters, such as cardiac EGMs. The electrodes used by IMDs to sense patient signals or parameters are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic plc. Some IMDs that do not provide therapy, e.g., implantable patient monitors, sense patient signals or parameters, such as cardiac EGMs. Examples of such IMDs are the Reveal LINQ™ and LINQ II™ Insertable Cardiac Monitor (ICMs), available from Medtronic, Inc., which may be inserted subcutaneously. Such IMDs may facilitate relatively longer-term monitoring of patients during normal daily activities and may periodically transmit collected data to a network service, such as the Medtronic Carelink™ Network.

IMDs may include a housing defining a cavity and configured to house processing circuitry configured to control the functioning of the IMD. The housing may include an electrically conductive portion defining the cavity, e.g., a titanium shell, and a dielectric cover, e.g., a sapphire cover, configured to enclose processing circuitry within the cavity. The IMD may also include electrodes (e.g., one or more electrodes), positioned on an outer surface of the dielectric cover and/or shell and connected to the processing circuitry, the processing circuitry being configured to monitor a physiological parameter of a patient via the electrode. The IMD may also include one or more components or devices, such as an antenna configured to send and receive information via electromagnetic radiation (e.g., wireless communication radio waves, such as according to the Bluetooth® protocol) or a or a sensor such as an optical sensor configured to detect, monitor, and/or sense a different parameter, which may also be a physiological parameter of the patient, a parameter of the environment external to the IMD, or any other suitable parameter.

The electrodes of the IMD are configured to be in contact with tissue and/or fluids of the patient in order to monitor the physiological parameter of a patient. In some examples, the electrodes comprise an anode and a cathode configured to be in contact with tissue and/or fluids of the patient and separated by a particular distance. If in electrical contact with tissue and/or fluids of the patient, the electrically conductive portion of the housing, while not in electrical contact with the electrodes, are in relatively close proximity to the electrodes, and may provide an electrical conduction path having a reduced electrical resistance (relative to patient tissue and/or fluids) between the electrodes. This condition could effectively “short” the electrodes and cause erroneous and/or missed measurements. For example, the electrically conductive portion of the housing may effectively be a conductor between tissue and/or fluids at the positions of the electrodes and cause the tissue and/or fluids at those positions to be at the same electrical potential and/or voltage when they otherwise would not be, and which may “block” biopotentials from being sensed by the electrodes.

Additionally, if a portion of an antenna of the IMD on the outer surface of the dielectric cover is in contact with surrounding tissue and/or fluids of the patient, the conductivity of the surrounding tissue and/or fluids may change and/or reduce the electrical current in the antenna caused by the communication radio waves and degrade the communication signal.

In order to prevent shorting of the electrodes (and improve communication signals), an IMD may include a biocompatible electrical insulator disposed on an outer surface of the electrically conductive portion and the dielectric cover, the biocompatible electrical insulator being configured to electrically isolate the conductive portion of the housing (and an antenna) from electrical contact with surrounding tissue and/or fluids. In some examples, the biocompatible electrical insulator may comprise a parylene, e.g., a parylene compound, parylene C, parylene N, or any suitable parylene.

Precise depositing and/or coating of the biocompatible electrical insulator may improve sensing of the physiological parameter of the patient via the electrodes, improve communication signals, and improve sensing of other parameters via other sensors (e.g., optical sensors) of the IMD, as well as improve the aesthetic look of the IMD, e.g., with clean, precise edges between exposed surfaces of the IMD and surfaces with biocompatible electrical insulator as opposed to ragged edges. However, precision deposition may be expensive, time consuming, and complex. It may be cheaper, easier, faster, and simpler to dispose the biocompatible electrical insulator over then entire surface of the IMD and precisely remove portions of the biocompatible electrical insulator from surface of the IMD, e.g., the electrodes and surfaces corresponding to other sensors.

In accordance with the systems, devices, and methods disclosed herein, a method of manufacturing an IMD includes disposing a biocompatible electrical insulator on outer surfaces of the IMD and precisely removing portions of the biocompatible electrical insulator from portions of the surface area of the IMD via ablating at least a portion of the biocompatible electrical insulator. In some examples, the methods and devices disclosed herein include masking portions of the IMD with a maskant before disposing the biocompatible electrical insulator on the IMD, and scoring the biocompatible electrical insulator via ablation in order to cleanly peel the maskant and corresponding biocompatible electrical insulator from the IMD. In other examples, the methods and devices disclosed herein include directly ablating one or more portions of the biocompatible electrical insulator to expose surfaces underneath, such as outer surfaces of the electrodes and/or portions corresponding to sensors housed within the housing of the IMD. In some examples, the methods and devices include texturing a surface of the IMD via the ablation energy, such as a surface of the electrodes, in addition to ablating the biocompatible electrical insulator to expose the textured surface. In some examples, texturing a surface of the electrodes may improve and/or enhance a surface roughness of the electrodes, e.g., via increasing a surface area of the electrodes. In some examples, texturing a surface of the IMD via ablation energy may create and/or form one or more of the electrodes.

The devices, systems, and techniques of this disclosure may provide one or more advantages and benefits. For instance, an IMD comprising a precisely positioned and/or removed biocompatible electrical insulator may provide improved physiological parameter monitoring and/or sensing signals, improved communications (speed, reliability, bandwidth, range, or the like), or reduced manufacturing cost, time, and complexity.

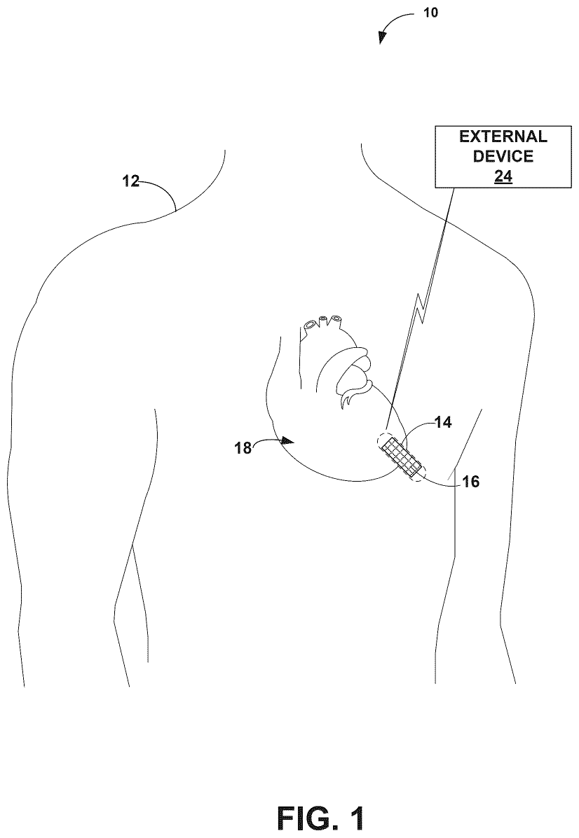

FIG. 1 is a conceptual drawing illustrating an example medical system 10 in conjunction with a patient 12 according to various examples described in this disclosure. The systems, devices, and methods described in this disclosure may include examples configurations of a biocompatible electrical insulator 16 disposed on an IMD 14, as illustrated and described with respect to FIG. 1. For purposes of this description, knowledge of cardiovascular anatomy and functionality is presumed, and details are omitted except to the extent necessary or desirable to explain the context of the techniques of this disclosure. System 10 includes IMD 14 having biocompatible electrical insulator 16, implanted at or near the site of a heart 18 of a patient 12 and an external computing device 24. The systems, devices, and methods described herein may provide infection control and migration control of IMD 14.

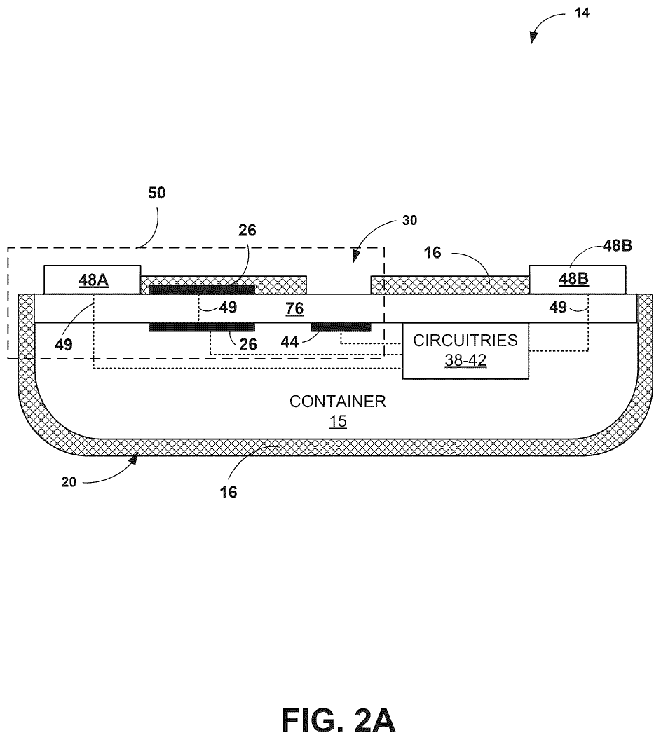

FIG. 2A is a conceptual cross-sectional side-view diagram illustrating an example configuration of the IMD 14 and biocompatible electrical insulator 16 of medical system 10 of FIG. 1, and FIG. 2B is a conceptual perspective view diagram illustrating the example configuration of the IMD 14 and biocompatible electrical insulator 16 of FIG. 2A, according to various examples described in this disclosure. FIGS. 1-2B are described concurrently below.

The example techniques may be used with IMD 14, which may be in wireless communication with at least one of external device 24 and other devices not pictured in FIG. 1. In some examples, IMD 14 is implanted outside of a thoracic cavity of patient 12 (e.g., subcutaneously in the pectoral location illustrated in FIG. 1). IMD 14 may be positioned near the sternum near or just below the level of the heart of patient 12, e.g., at least partially within the cardiac silhouette. IMD 14 includes a plurality of electrodes 48 (FIG. 2) and is configured to sense a cardiac electrogram (EGM) via the plurality of electrodes. In some examples, IMD 14 takes the form of the LINQ™ or LINQ II™ ICM, or another ICM similar to, e.g., a version or modification of, the LINQ™ or LINQ II™ ICM. Although described primarily in the context of examples in which IMD 14 is an ICM, in various examples, IMD 14 may represent a cardiac monitor, a defibrillator, a cardiac resynchronization pacer/defibrillator, a pacemaker, an implantable pressure sensor, a neurostimulator, or any other implantable or external medical device.

In some examples, IMD 14 is defined by a length L, a width Wand thickness or depth D and is in the form of an elongated rectangular prism wherein the length L is much larger than the width W, which in turn is larger than the depth D, as illustrated in FIG. 2B below. In one example, the geometry of the IMD 14—in particular a width W greater than the depth D—is selected to allow IMD 14 to be inserted under the skin of the patient using a minimally invasive procedure and to remain in the desired orientation during insert. For example, IMD 14 may include a radial asymmetry (notably, a rectangular shape) along the longitudinal axis that maintains the device in the proper orientation following insertion.

For example, in one example the spacing between electrode 48A and electrode 48B may range from 30 millimeters (mm) to 55 mm, 35 mm to 55 mm, and from 40 mm to 55 mm and may be any range or individual spacing from 25 mm to 60 mm. In another example the spacing between electrode 48A and electrode 48B may range from 15 mm to 30 mm, 17 mm to 28 mm, and from 20 mm to 28 mm and may be any range or individual spacing from 12 mm to 30 mm. In addition, IMD 14 may have a length L that ranges from 30 mm to about 70 mm. In other embodiments, the length L may range from 40 mm to 60 mm, 45 mm to 60 mm and may be any length or range of lengths between about 30 mm and about 70 mm. In some examples, IMD 14 may have a length L that ranges from 15 mm to about 35 mm, or from 20 mm to 30 mm, 22 mm to 30 mm and may be any length or range of lengths between about 15 mm and about 35 mm.

In addition, the width W of a major surface of IMD 14, e.g., dielectric cover 76 in the example shown, may range from 3 mm to 10 mm and may be any single or range of widths between 3 mm and 10 mm, or may range from 1.5 mm to 5 mm and may be any single or range of width between 1.5 mm and 5 mm. The thickness of depth D of IMD 14 may range from 2 mm to 9 mm, or from 1.5 mm to 4.5 mm. In other embodiments, the depth D of IMD 14 may range from 2 mm to 5 mm and may be any single or range of depths from 2 mm to 9 mm, or may range from 1 mm to 2.5 mm and may be any single or range of depths from 1 mm to 4.5 mm. In addition, IMD 14 according to an example of the present invention has a geometry and size designed for ease of implant and patient comfort. Examples of IMD 14 described in this disclosure may have a volume of 3 cubic centimeters (cm) or less, 1.5 cubic cm or less or any volume between 3 and 1.5 cubic centimeters, or may have a volume of 1.5 cubic centimeters (cm) or less, 0.75 cubic cm or less or any volume between 1.5 and 0.75 cubic centimeters.

External device 24 may be a computing device with a display viewable by the user and an interface for providing input to external device 24 (i.e., a user input mechanism). In some examples, external device 24 may be a notebook computer, tablet computer, workstation, one or more servers, cellular phone, personal digital assistant, or another computing device that may run an application that enables the computing device to interact with IMD 14. External device 24 is configured to communicate with IMD 14 and, optionally, another computing device (not illustrated in FIG. 1), via wireless communication. External device 24, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

External device 24 may be used to configure operational parameters for IMD 14. External device 24 may be used to retrieve data from IMD 14. The retrieved data may include values of physiological parameters measured by IMD 14, indications of episodes of arrhythmia or other maladies detected by IMD 14, and physiological signals recorded by IMD 14. For example, external device 24 may retrieve cardiac EGM segments recorded by IMD 14, e.g., due to IMD 14 determining that an episode of arrhythmia or another malady occurred during the segment, or in response to a request to record the segment from patient 12 or another user. In some examples, one or more remote computing devices may interact with IMD 14 in a manner similar to external device 24, e.g., to program IMD 14 and/or retrieve data from IMD 14, via a network.

In various examples, IMD 14 may include one or more additional sensors and sensor circuits configured to sense a particular physiological or neurological parameter associated with patient 12, or may comprise a plurality of sensor circuits, which may be located at various and/or different positions relative to patient 12 and/or relative to each other and may be configured to sense one or more physiological parameters associated with patient 12.

For example, IMD 14 may include a sensor operable to sense a body temperature of patient 12 in a location of the IMD 14, or at the location of the patient where a temperature sensor coupled by a lead to IMD 14 is located. In another example, IMD 14 may include a sensor configured to sense motion, such as steps taken by patient 12 and/or a position or a change of posture of patient 12. In various examples, IMD 14 may include a sensor that is configured to detect breaths taken by patient 12. In various examples, IMD 14 may include a sensor configured to detect heartbeats of patient 12. In various examples, IMD 14 may include a sensor that is configured to measure systemic blood pressure of patient 12. In some examples, IMD 14 may include a sensor that is configured to measure an oxygenation of blood of patient 12, e.g., an optical sensor and optical light source such as a infrared detector and infrared LED.

In some examples, one or more of the sensors comprising IMD 14 may be implanted within patient 12, that is, implanted below at least the skin level of the patient. In some examples, one or more of the sensors of IMD 14 may be located externally to patient 12, for example as part of a cuff or as a wearable device, such as a device imbedded in clothing that is worn by patient 12. In various examples, IMD 14 may be configured to sense one or more physiological parameters associated with patient 12, and to transmit data corresponding to the sensed physiological parameter or parameters to the external device 24, as represented by the lightning bolt coupling IMD 14 to the external device 24.

Transmission of data from IMD 14 to external device 24 in various examples may be performed via wireless transmission, using for example any of the formats for wireless communication described above. In various examples, IMD 14 may communicate wirelessly to an external device (e.g., an instrument or instruments) other than or in addition to external device 24, such as a transceiver or an access point that provides a wireless communication link between IMD 14 and a network. Examples of communication techniques used by any of the devices described above with respect to FIG. 1 may include radiofrequency (RF) telemetry, which may be an RF link established via Bluetooth®, Wi-Fi, or medical implant communication service (MICS).

In some examples, system 10 may include more or fewer components than depicted in FIG. 1. For example, in some examples, system 10 may include multiple additional IMDs, such as implantable pacemaker devices or other IMDs, implanted within patient 12. In these examples, IMD 14 may function as a hub device for the other IMDs. For example, the additional IMDs may be configured to communicate with the IMD 14, which would then communicate to the external device 24, such as a user's smartphone, via a low-energy telemetry protocol. IMD 14 may provide a theoretically infinite energy capacity, in that IMD 14 may not need to be replaced or otherwise removed. Accordingly, IMD 14 may provide the ability to more-frequently telemeter information, as well as more-active titration of therapies.

For the remainder of the disclosure, a general reference to a medical device system may refer collectively to include any examples of medical device system 10, a general reference to IMD 14 may refer collectively to include any examples of IMD 14, a general reference to sensor circuits may refer collectively to include any examples of sensor circuits of IMD 14, and a general reference to an external device may refer collectively to any examples of external device 24.

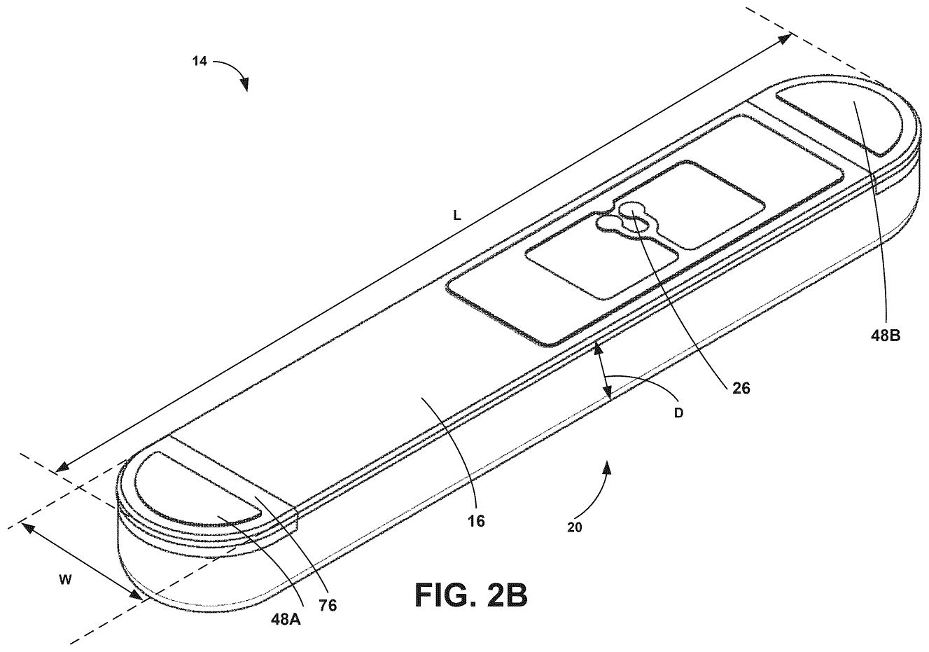

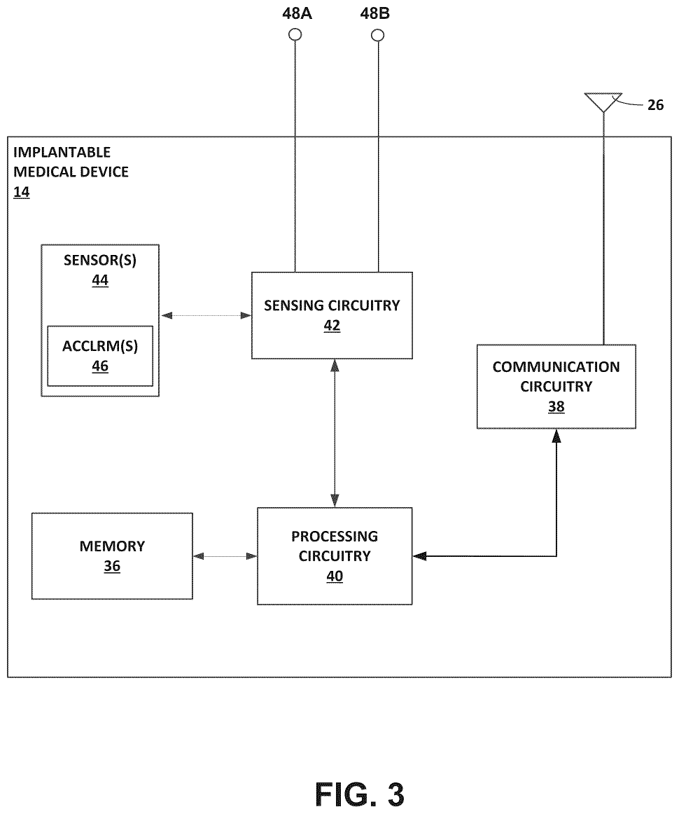

In the examples shown in FIGS. 2A-2B, IMD 14 may include a leadless, subcutaneously implantable monitoring device having a container 15 and a dielectric cover 76. Electrode 48A and electrode 48B (collectively “electrodes 48”) may be formed or placed on an outer surface of dielectric cover 76. In other examples, one or more electrodes 48 may be formed or placed on an outer surface of container 15 (not shown). Circuitries 36-42, described below with respect to FIG. 3, may be formed or placed on an inner surface of dielectric cover 76, or within container 15. In some examples, antenna 26 is formed or placed on the inner surface of dielectric cover 76. In other examples, antenna 26 is formed or placed on the outer surface of dielectric cover 76, and in other examples, antenna 26 may be formed or placed at least partially on the inner surface and partially on the outer surface of dielectric cover 76. In some examples, sensor 44 is formed or placed on the inner surface of dielectric cover 76, or within container 15.

In some examples, dielectric cover 76 may be positioned over an open container 15 such that container 15 and dielectric cover 76 form housing 20 and enclose circuitries 36-42, sensor 44, (and in some cases antenna 26) and protect the circuitries from fluids such as body fluids. One or more of circuitries 36-42 and/or sensor 44 may be formed on the inner side of dielectric cover 76, such as by using flip-chip technology. Dielectric cover 76 may be flipped onto a container 15. When flipped and placed onto container 15, the components of IMD 10 formed on the inner side of dielectric cover 76 may be positioned in a gap defined by container 15.

Electrodes 48, sensor 44, and antenna 26 (when placed or formed on the outer surface of dielectric cover 76) may be electrically connected to sensing circuitry 42 and communication circuitry 38 (illustrated in FIG. 3), respectively, through one or more vias 49 formed through dielectric cover 76. Dielectric cover 76 may be formed of sapphire (i.e., corundum), glass, and/or any other suitable insulating material. Container 15 may be formed from any suitable material configured to house circuitries 36-42, support and mate with cover 26 to isolate circuitries 36-42 for contact with tissue and/or fluids of patient 12, and to be implantable within patient 12. In some examples, container 15 may house a battery or other power source. In some examples, container 15 may also be electrically conductive. For example, container 15 may be formed from titanium or any other suitable material (e.g., a biocompatible material). Electrodes 48 may be formed from any of stainless steel, titanium, platinum, iridium, or alloys thereof. In addition, electrodes 48 may be coated with a material such as titanium nitride or fractal titanium nitride, although other suitable materials and coatings for such electrodes may be used.

Biocompatible electrical insulator 16 is disposed on housing 20. In the example shown, biocompatible electrical insulator 16 is disposed on portions of dielectric cover 76 and the outer surface of container 15. In some examples, biocompatible electrical insulator 16 may be disposed on all or a portion of any outer surface of IMD 14 and/or housing 20, except for outer surfaces of electrodes 48 and/or portions of dielectric cover 76 corresponding to sensor 44, e.g., ap 30 (FIG. 2A). For example, biocompatible electrical insulator 16 may be disposed on all or a portion of container 15, dielectric cover 76, or at least a portion of both container 15 and dielectric cover 76. In some examples, biocompatible electrical insulator 16 may be disposed on greater than or equal to 20% of the surface area of housing 20, on greater than or equal to 50% of the surface area of housing 20, on greater than or equal to 75% of the surface area of housing 20, on greater than or equal to 90% of the surface area of housing 20, or any suitable surface area of housing 20. In some examples, biocompatible electrical insulator 16 may be disposed on substantially all of housing 20 surface area that is not a sensor electrode, e.g., electrode 48A or 48B. In some examples, biocompatible electrical insulator 16 may be disposed on greater than 55% of housing 20 surface area that is not electrode 48A or 48B and in some other examples, biocompatible electrical insulator 16 may be disposed on greater than 90% of housing 20 surface area that is not electrode 48A or 48B.

In some examples, biocompatible electrical insulator 16 may be disposed on at least a portion of housing 20 surface area by vacuum depositing a coating of biocompatible electrical insulator 16 on housing 20. For example, biocompatible electrical insulator 16 may conform to the shape of housing 20 and be configured to cover over, adhere to, and/or attach to the outer surface of housing 20 and/or outer surfaces of components on the outer surface of housing 20, e.g., antenna 26, and electrodes 48, while also being configured to be removable to portions of housing 20 and electrodes 48, e.g., without damaging electrodes 48. In some examples, biocompatible electrical insulator 16 may be disposed (e.g., deposited, laminated, coated, or the like) on a portion of the outer surface of dielectric cover 76 to as to cover and/or encapsulate antenna 26, or at least a portion of antenna 26 disposed on the outer surface of dielectric cover 76, and biocompatible electrical insulator 16 may be alternatively or additionally disposed on all of the outer surface of container 16, e.g., to cover and/or encapsulate container 15 so as to electrically isolate container 15 from electrical contact with tissue and/or fluid of patient 12.

Biocompatible electrical insulator 16 may be configured to not interfere with the efficacy of IMD 14, e.g., electrodes 48, receiving a physiological signal. In the example shown, biocompatible electrical insulator 16 is disposed on surface areas of dielectric cover 76 and container 15 not including electrodes 48. In some examples, biocompatible electrical insulator 16 is configured to improve the efficacy of IMD 14. For example, biocompatible electrical insulator 16 may be configured to improve antenna 26 receiving and/or sending communication signals by encapsulating and electrically isolating at least a portion of antenna 26 disposed on an outer surface of dielectric cover 76 from tissue and/or fluids of patient 12.

In some examples, biocompatible electrical insulator 16 may be disposed on housing 20 in a pattern. For example, biocompatible electrical insulator 16 may be disposed on a first area of housing 20 and not disposed on a second area of housing 20. In some examples, biocompatible electrical insulator 16 may be ablated, e.g., via laser ablation, to form the pattern. For example, a portion of biocompatible electrical insulator 16 may be removed via ablation to expose outer surfaces of electrodes 48 and/or a portion of the outer surface of dielectric cover 76 and/or a portion of the outer surface of container 15. In other examples, a portion of biocompatible electrical insulator 16 may be removed via ablation to score biocompatible electrical insulator 16, e.g., for an easy, clean tear of biocompatible electrical insulator 16 to remove biocompatible electrical insulator 16 via peeling. For example, prior to disposing biocompatible electrical insulator 16 on housing 20 and electrodes 48, a maskant may be disposed on portions of housing 20 and electrodes. Biocompatible electrical insulator 16 may then be scored via laser ablation adjacent and/or corresponding to the maskant, and the maskant and corresponding portion of biocompatible electrical insulator 16 disposed on the maskant may be peeled to be removed from electrodes 48 and/or portions of housing 20.

In some examples, biocompatible electrical insulator 16 may be disposed with a thickness, e.g., at least 25 micrometers thick, at least 100 micrometers thick, at least 1 millimeter thick, at least 5 millimeters thick, at least 10 millimeters thick, or any suitable thickness. In some examples, biocompatible electrical insulator 16 may be disposed on housing 20 having a substantially uniform layer thickness. In other examples, biocompatible electrical insulator 16 may be disposed on housing 20 having a layer thickness that varies, e.g., biocompatible electrical insulator 16 may be disposed on container 15 with a thicker layer thickness than biocompatible electrical insulator 16 disposed on dielectric cover 76, or vice versa. In some examples, biocompatible electrical insulator 16 may comprise parylene, e.g., a parylene compound, parylene C, parylene N, or any suitable parylene.

FIG. 3 is a functional block diagram illustrating an example configuration of the implantable medical device (IMD) and of the medical system of FIG. 1. In the illustrated example, IMD 14 includes processing circuitry 40, memory 36, communication circuitry 38, communication antenna 26, sensing circuitry 42, sensor(s) 44, accelerometer(s) 46, and electrodes 48A and 48B (collectively, “electrodes 48”). Although the illustrated example includes two electrodes 48, IMDs including or coupled to one electrode 48, or more than two electrodes 48, may implement the techniques of this disclosure in some examples.

Processing circuitry 40 may include fixed function circuitry and/or programmable processing circuitry. Processing circuitry 40 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 40 may include multiple components, such as any combination of one or more microprocessors, one or more controllers, one or more DSPs, one or more ASICs, or one or more FPGAs, as well as other discrete or integrated logic circuitry. The functions attributed to processing circuitry 40 herein may be embodied as software, firmware, hardware or any combination thereof.

Sensing circuitry 42 is coupled to electrodes 48 and is configured to monitor one or more physiological parameters of a patient. Sensing circuitry 42 may sense signals from electrodes 48, e.g., to produce a cardiac EGM, in order to facilitate monitoring the electrical activity of the heart. Sensing of a cardiac EGM may be done to determine heart rates or heart rate variability, or to detect arrhythmias (e.g., tachyarrhythmias or bradycardia). Sensing circuitry 42 may additionally monitor impedance or other electrical phenomena via electrodes 48. Sensing circuitry 42 also may monitor signals from sensors 44, which may include one or more accelerometers 46, pressure sensors, and/or optical sensors, as examples. In some examples, sensing circuitry 42 may include one or more filters and amplifiers for filtering and amplifying signals received from electrodes 48 and/or sensors 44. In some examples, sensing circuitry 42 may sense or detect physiological parameters, such as heart rate, blood pressure, respiration, and other physiological parameters associated with a patient.

Sensing circuitry 42 and/or processing circuitry 40 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 42 may include a rectifier, filter, amplifier, comparator, and/or analog-to-digital converter, in some examples. In some examples, sensing circuitry 42 may output an indication to processing circuitry 40 in response to sensing of a cardiac depolarization. In this manner, processing circuitry 40 may receive detected cardiac depolarization indicators corresponding to the occurrence of detected R-waves and P-waves in the respective chambers of heart. Processing circuitry 40 may use the indications of detected R-waves and P-waves for determining inter-depolarization intervals, heart rate, and detecting arrhythmias, such as tachyarrhythmias, bradyarrhythmias, and asystole.

Sensing circuitry 42 may also provide one or more digitized cardiac EGM signals to processing circuitry 40 for analysis, e.g., for use in cardiac rhythm discrimination. In some examples, processing circuitry 40 may store the digitized cardiac EGM in memory 36. Processing circuitry 40 of IMD 14, and/or processing circuitry of another device that retrieves data from IMD 14, may analyze the cardiac EGM.

Communication circuitry 38 may include any suitable hardware, firmware, software or any combination thereof for communicating with another device, such as external device 24, another networked computing device, or another IMD or sensor. Under the control of processing circuitry 40, communication circuitry 38 may receive downlink telemetry from, as well as send uplink telemetry to external device 24 or another device with the aid of an internal or external antenna, e.g., antenna 26. In addition, processing circuitry 40 may communicate with a networked computing device via an external device (e.g., external device 24 of FIG. 1) and a computer network, such as the Medtronic CareLink® Network. Antenna 26 and communication circuitry 38 may be configured to transmit and/or receive signals via inductive coupling, electromagnetic coupling, Near Field Communication (NFC), Radio Frequency (RF) communication, Bluetooth, Wi-Fi, or other proprietary or non-proprietary wireless communication schemes. Communication antenna 26 may telemeter data at a high frequency, such as around 2.4 gigahertz (GHz).

In some examples, memory 36 includes computer-readable instructions that, when executed by processing circuitry 40, cause IMD 14 and processing circuitry 40 to perform various functions attributed to IMD 14 and processing circuitry 40 herein. Memory 36 may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media. Memory 36 may store, as examples, programmed values for one or more operational parameters of IMD 14 and/or data collected by IMD 14, e.g., posture, heart rate, activity level, respiration rate, and other parameters, as well as digitized versions of physiological signals sensed by IMD 14, for transmission to another device using communication circuitry 38.

In the illustrated example, IMD 14 includes processing circuitry 40 and an associated memory 36, sensing circuitry 42, one or more sensors 44, and the communication circuitry 38 coupled to antenna 26 as described above. However, IMD 14 need not include all of these components, or may include additional components.

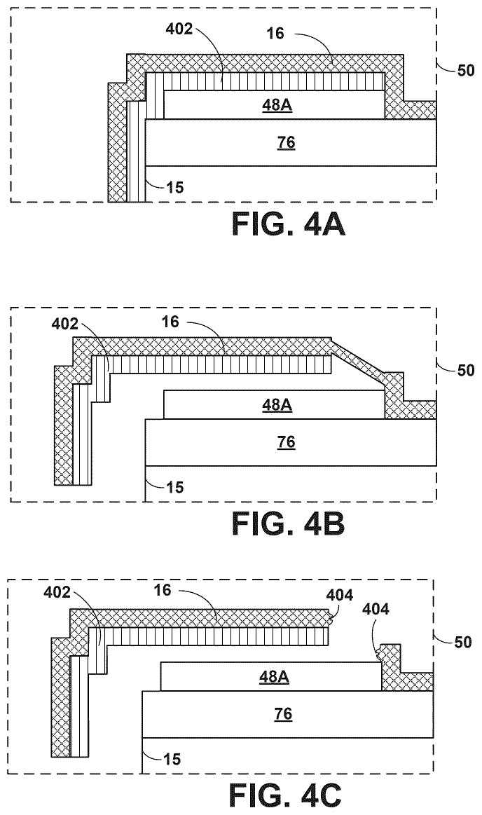

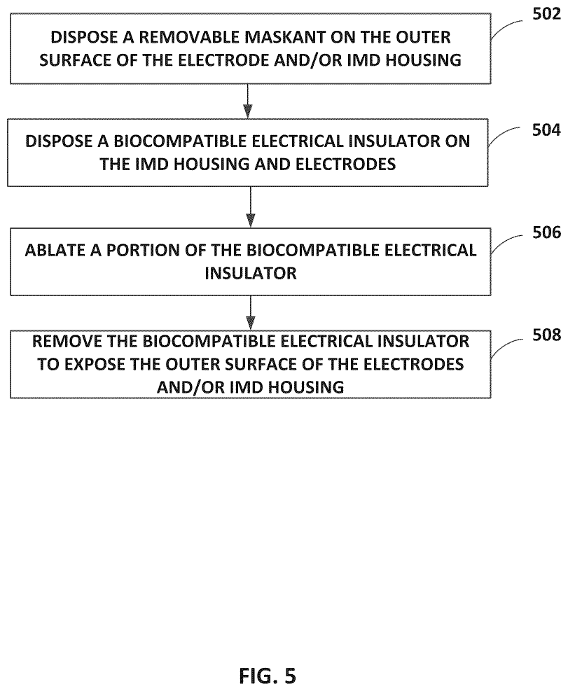

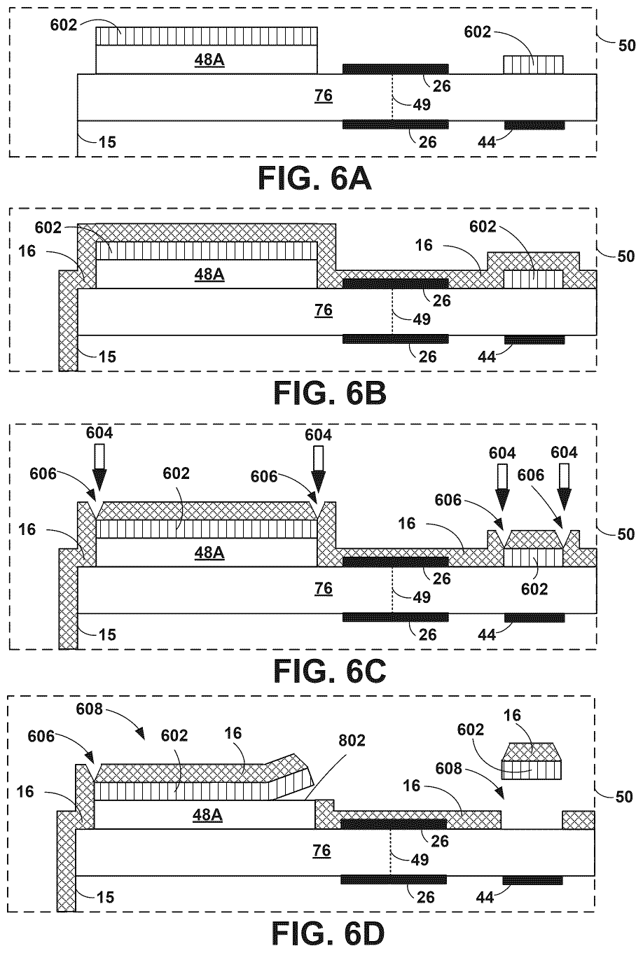

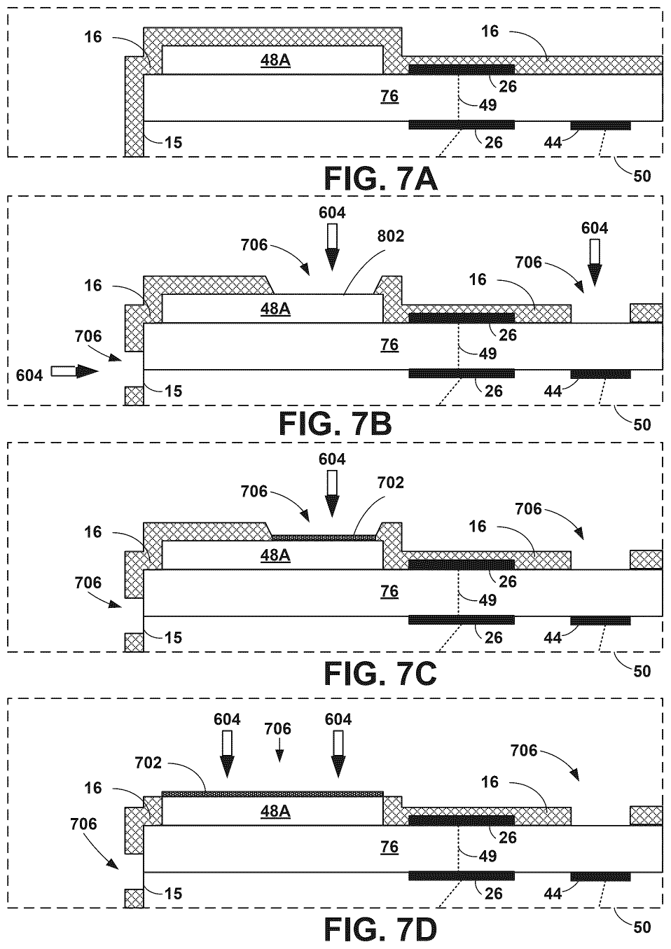

FIGS. 4A-4C are a series of conceptual cross-sectional side-views illustrating configurations of a portion 50 of IMD 14 at different method steps of a masking method of removing biocompatible electrical insulator 16 from electrodes 48 and/or portions of housing 20 without ablating a portion of biocompatible electrical insulator 16. The portion of IMD 14 shown is indicated in FIG. 2A by portion 50. The method steps illustrated by FIGS. 4A-4C result in a ragged tear and ragged boundary of biocompatible electrical insulator 16, and are described and shown to contrast with the ablation methods described below, e.g., FIG. 5, FIGS. 6A-6D and FIGS. 7A-7D.

A mask 402 may be disposed on a portion of housing 20 and electrodes 48 intended to be exposed and not covered by biocompatible electrical insulator 16, and biocompatible electrical insulator 16 may then be disposed over the mask 402 as shown in FIG. 4A. The mask may be removable from housing 20 and electrodes 48, and may be pulled and/or peeled from the outer surfaces of housing 20 and electrodes 48 as shown in FIG. 4B. The resulting tear leaves a ragged edges 404 of the biocompatible electrical insulator 16 (FIG. 4C), and can sometimes result in portions of biocompatible electrical insulator 16 tearing/peeling away from surfaces that are intended to remain covered, or portions of biocompatible electrical insulator 16 being left on surfaces that are intended to be exposed, e.g., inaccurate and imprecise removal of biocompatible electrical insulator 16.

FIG. 5 is a flow diagram of an example method of manufacturing an implantable medical device, according to various examples described in this disclosure. Although example method of FIG. 5 is described with respect to medical system 10, IMD 14, biocompatible electrical insulator 16, handle 100, and mount 102 of FIGS. 1-9, the example technique of FIG. 5 may be performed using any system including an implantable medical device and a biocompatible electrical insulator described herein. The method of FIG. 5 may be performed by any suitable user, such as a manufacturer, and the like. The method of FIG. 5 is described in conjunction with FIGS. 6A-6D and FIGS. 7A-7D. FIGS. 6A-6D and FIGS. 7A-7D are conceptual cross-sectional side-views illustrating configurations of the portion 50 of IMD 14 at different steps of the method of FIG. 5.

A manufacturer may dispose biocompatible electrical insulator 16 on an outer surface of housing 20 and to cover an outer surface of electrodes 48 positioned on the outer surface of housing 20 (502). For example, the manufacturer may dispose biocompatible electrical insulator 16 on any or all of dielectric cover 76, container 15, electrode 48A, and/or antenna 26, or on any layer intermediate layer, e.g., maskant In some examples, the manufacturer may dispose of biocompatible electrical insulator 16 on the housing of IMD 14 by placing IMD 14 in a vacuum deposition chamber, and coating housing 20 and electrodes 48 with biocompatible electrical insulator 16 via vacuum deposition.

A manufacturer may dispose a removable maskant 602 on an outer surface of electrodes 48 and or housing 20 (502). For example, the manufacturer may dispose removable maskant 602 on any or all of dielectric cover 76, container 15, electrodes 48, and/or antenna 26. In the example shown in FIG. 6A, the manufacturer may dispose maskant 602 on electrode 48A and a portion of dielectric cover 76 corresponding to sensor 44. In some examples, the manufacturer may dispose maskant 602 on IMD 14 via coating, vacuum deposition, spray coating, or any suitable method. Maskant 602 may be configured to adhere to outer surfaces of any or all of dielectric cover 76, container 15, electrodes 48, and/or antenna 26 with sufficient adhesive to handle IMD 14 and/or subsequently dispose biocompatible electrical insulator 16 on IMD 14 and maskant 602, while also being able to be removed and/or released from the outer surfaces of dielectric cover 76, container 15, electrodes 48, and/or antenna 26 without damaging outer surfaces of dielectric cover 76, container 15, electrodes 48, and/or antenna 26.

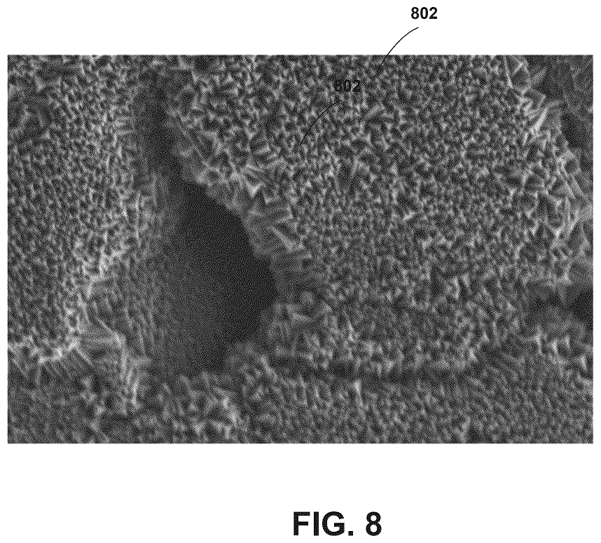

For example, electrodes 48 may comprise a titanium nitride textured and/or structured surface, e.g., a titanium nitride (TiN) surface having an engineered surface structure. FIG. 8 is a scanning electron microscopy (SEM) image of an example TiN surface structure 802, according to various examples described in this disclosure. Electrode 48A may comprise a titanium foil with TiN surface structure 802 applied to the titanium foil. In some examples, the TIN surface structure 802 comprises micro- and/or nano-sized features, e.g., features having heights, depths, lengths, and/or widths on a micrometer and/or nanometer scale. Maskant 602 may be configured to adhere to, and be removed from, electrode 48A having TiN surface structure 802 without removing and/or delaminating portions of surface structure 802 from electrode 48A, without leaving a residue that changes TiN surface structure 802, and/or otherwise damaging and/or changing TiN surface structure 802. For example, maskant 602 may include a UV cure adhesive, a molded silicone, a tape (e.g., such as a Kapton tape), or any suitable removable material. In some examples, disposing maskant 602 may be used in conjunction with ablating biocompatible electrical insulator 16 and peeling or otherwise removing maskant 602 to precisely remove biocompatible electrical insulator 16. In other examples, disposing maskant 602 at step (502) is optional and may not be used, e.g., in conjunction with ablating (e.g., directly ablating) biocompatible electrical insulator 16 to precisely remove biocompatible electrical insulator 16.

A manufacturer may dispose biocompatible electrical insulator 16 on an outer surface of housing 20 and to cover an outer surface of electrodes 48 positioned on the outer surface of housing 20 (504). For example, the manufacturer may dispose biocompatible electrical insulator 16 on any or all of dielectric cover 76, container 15, electrode 48A, and/or antenna 26 (FIGS. 6B and 7A), or on any layer intermediate layer, e.g., maskant 602 (FIG. 6B). In some examples, the manufacturer may dispose of biocompatible electrical insulator 16 on the housing of IMD 14 by placing IMD 14 in a vacuum deposition chamber, and coating housing 20 and electrodes 48 with biocompatible electrical insulator 16 via vacuum deposition. In some examples, biocompatible electrical insulator 16 may comprise any parylene, e.g., a parylene compound, parylene C, parylene N, or any suitable parylene.

The manufacturer may ablate a portion 706 of biocompatible electrical insulator 16 (506). For example, the manufacturer may ablate one or more portions 606 and/or 706 (FIGS. 6C and 7B) of biocompatible electrical insulator 16 using ablation energy 604. In some examples, ablating the one or more portions 606 of biocompatible electrical insulator 16 comprises scoring and/or perforating portions of biocompatible electrical insulator 16 (FIG. 6C), e.g., in preparation for tearing and peeling biocompatible electrical insulator 16 with a precise, clean edge or boundary. In other examples, ablating the one or more portions 606 of biocompatible electrical insulator 16 comprises ablating and removing (e.g., directly ablating) an entire portion 706 or area of biocompatible electrical insulator 16, e.g., ablation and removal in a single step.

In some examples, the ablation energy 604 may be laser ablation energy. For example, ablating the portions 606 and/or 706 of the biocompatible electrical insulator may include laser ablation with at least one of an ultraviolet fiber laser, a femtosecond pulsed laser, an excimer laser, or any suitable ablation energy 604.

In some examples, the manufacturer may ablate a portion 706 of biocompatible electrical insulator 16 without damaging a surface of IMD 14, e.g., without damaging engineered surface structure 802 if electrodes 48A (FIG. 7B). In the example shown in FIG. 7B, the manufacturer may ablate and remove portion 706 of biocompatible electrical insulator 16 from the outer surface of electrode 48A including surface structure 802 without damaging surface structure 802, e.g., without removing and/or delaminating portions of surface structure 802 from electrode 48A, without leaving a residue that changes TiN surface structure 802, and/or otherwise damaging and/or changing TiN surface structure 802.

In some examples, the method of FIG. 5 may enable IMD 14 to be manufactured without a TiN surface structure 802. For example, at (506), the manufacturer may ablate and remove biocompatible electrical insulator 16 and concurrently texture the outer surface of electrode 48A. For example, electrode 48A may comprise a titanium electrode, and as shown in FIG. 7C, the manufacture may ablate biocompatible electrical insulator 16 to remove biocompatible electrical insulator 16 from the outer surface of electrode 48A and simultaneously texture (e.g., add a surface structure to) the outer surface of electrode 48A with the ablation energy 604 to form surface texture 702. Texturing the outer surface of electrode 48A may enable IMD 14 to omit a TiN surface and/or surface treatment of electrode 48A. In other examples, the manufacturer may ablate and remove biocompatible electrical insulator 16, and then subsequently texture the outer surface of electrode 48A with ablation 604 to form textured surface 702 as a separate step after removal of biocompatible electrical insulator 16 (FIG. 7D).

In some examples, the manufacturer may ablate a portion 706 of biocompatible electrical insulator 16 having a reduced transmission of the ablation energy 604. For example, dielectric cover 76 may have a relatively high transmission for the ablation energy 604, and when ablating a portion 706 corresponding to sensor 44 (FIG. 7B), the ablation energy 604 may damage components within container 15, e.g., processing circuitry 38-42, antenna 26, vias 49, or the like. Biocompatible electrical insulator 16 may have a reduced transmission of ablation energy 604, e.g., biocompatible electrical insulator 16 may be at least partially absorbing of ablation energy 604 and/or include absorbing materials and/or particles configured to reduce the amount of energy transmitted through biocompatible electrical insulator 16, and also through dielectric cover 76. Alternatively or additionally, dielectric cover 76 may be at least partially absorbing of ablation energy 604 and/or include absorbing materials and/or particles configured to reduce the amount of energy transmitted through dielectric cover 76.

The manufacturer may remove biocompatible electrical insulator 16 to expose the outer surface of electrodes 48 and/or IMD housing 20 (508). For example, as shown in FIG. 6D, the manufacturer may peel, lift, or otherwise remove portions 608 of maskant 602 and biocompatible electrical insulator 16 using ablated portions 606 (e.g., the scoring) to result in a precise (e.g., precisely and accurately positioned), clean (e.g., non-ragged) edge or boundary of biocompatible electrical insulator 16.

In some examples, the manufacturer may ablate and remove a plurality of portions 606, 608, and/or 706 of biocompatible electrical insulator 16 (e.g., FIGS. 6C, 6D, and 7B) sequentially or concurrently. For example, the manufacturer may remove biocompatible electrical insulator 16 via raster scanning laser ablation energy 604 via moving the ablation energy source and/or IMD 14, e.g., on rotary and/or translation stages, to ablate the plurality of portions 606, 608, and/or 706. In other examples, the manufacturer may remove biocompatible electrical insulator 16 concurrently to expose biocompatible electrical insulator 16 to laser ablation energy 604 in a pattern, e.g., corresponding to the areas of the portions 606, 608, and/or 706 to be scored and/or removed via ablation. In some examples, the manufacturer may ablate portions 706 of biocompatible electrical insulator 16 facing different directions, sequentially or concurrently. For example, the manufacturer may ablate portions 706 of biocompatible electrical insulator 16 facing directions that are least ninety degrees different from each other, e.g., as illustrated in FIG. 7B by portions 706 ablated from dielectric cover 76 and electrode 48A facing a different direction (e.g., by about 90 degrees) than portion 76 ablated from container 15. In some examples, the manufacturer may ablate portions 706 of biocompatible electrical insulator 16 from opposing surfaces, e.g., dielectric cover 76 and/or electrode 48A and also from container 15 on the side of IMD 14 facing the opposite direction from dielectric cover 76 and electrode 48A.

This disclosure includes the following non-limiting examples.

Example 1: A method of manufacturing an implantable medical device, the method including: disposing a biocompatible electrical insulator on an outer surface of a housing of the implantable medical device and to cover an outer surface of an electrode that is positioned on the outer surface of the housing; ablating a portion of the biocompatible electrical insulator; and removing the biocompatible electrical insulator to expose the outer surface of the electrode.

Example 2: The method of example 1, wherein the biocompatible electrical insulator comprises parylene.

Example 3: The method of example 1 or example 2, wherein the outer surface of the electrode comprises a titanium nitride (TiN) surface having an engineered surface structure, wherein ablating or removing the portion of the biocompatible electrical insulator does not damage the engineered surface structure.

Example 4: The method of example 3, further including: prior to disposing the biocompatible electrical insulator to cover the outer surface of the electrode, disposing a removable maskant on the outer surface of the electrode.

Example 5: The method of example 4, wherein ablating the portion of the biocompatible electrical insulator comprises scoring a first portion of the biocompatible electrical insulator adjacent to the removable maskant.

Example 6: The method of example 5, wherein removing the biocompatible electrical insulator comprises peeling the removable maskant and a second portion of the biocompatible electrical insulator from the outer surface of the electrode, wherein peeling the removable maskant from the outer surface of the electrode does not damage the engineered surface structure.

Example 7: The method of example 5 or example 6, wherein the removeable maskant comprises at least one of a UV cure adhesive, a molded silicone, or a Kapton tape.

Example 8: The method of any one of examples 1-7, wherein ablating the portion of the biocompatible electrical insulator comprises the removing the biocompatible electrical insulator to expose an outer surface of the electrode.

Example 9: The method of any one of examples 1-8, wherein ablating the portion of the biocompatible electrical insulator comprises laser ablation with at least one of an ultraviolet fiber laser, a femtosecond pulsed laser, or an excimer laser.

Example 10: The method of example 9, wherein the outer surface of the electrode comprises a titanium surface, wherein the method further comprises texturing the titanium surface via the laser ablation.

Example 11: The method of example 10, wherein the texturing occurs at the same time as ablating the portion of the biocompatible electrical insulator.

Example 12: The method of any one of examples 1-11, wherein the portion of the biocompatible electrical insulator is a first portion, wherein the housing comprises: an electrically conductive portion defining a cavity configured to receive processing circuitry, wherein the processing circuitry is configured to control functioning of the implantable medical device; and a dielectric cover configured to cover the cavity and enclose the processing circuitry, wherein the electrode is positioned on an outer surface of the dielectric cover and is connected to the processing circuitry, wherein the processing circuitry is configured to monitor a physiological parameter of a patient via the electrode, wherein disposing the biocompatible electrical insulator on the outer surface of the housing comprises disposing the biocompatible electrical insulator on an outer surface of the dielectric cover, the method further includes ablating a second portion of the biocompatible electrical insulator to expose at least a portion of the outer surface of the dielectric cover.

Example 13: The method of example 12, wherein the portion of the outer surface of the dielectric cover corresponds to a sensor housed within the cavity, wherein the biocompatible electrical insulator reduces a transmission of an ablation energy through the dielectric cover.

Example 14: The method of example 12 or example 13, wherein the at least the portion of the biocompatible electrical insulator is a first portion of the biocompatible electrical insulator, wherein the outer surface of the housing is a first outer surface of the housing, the method further comprising ablating a second portion of the biocompatible electrical insulator from a second portion of the housing.

Example 15: The method of example 14, wherein the second portion of the housing is facing a direction that is at least ninety degrees different than a direction that the first portion of the housing is facing.

Example 16: An implantable medical device including: a housing configured to house processing circuitry, wherein the processing circuitry is configured to control functioning of the implantable medical device, wherein the housing comprises: an electrically conductive portion defining a cavity configured to receive the processing circuitry; and a dielectric cover configured to cover the cavity and enclose the processing circuitry within the cavity; an electrode positioned on an outer surface of the dielectric cover and opposite the processing circuitry, wherein the processing circuitry is configured to monitor a physiological parameter of a patient via the electrode; and a biocompatible electrical insulator disposed on an outer surface of at least one of the electrically conductive portion or the dielectric cover, wherein a first portion of the biocompatible electrical insulator is ablated, and wherein a second portion of the biocompatible electrical insulator is removed to expose an outer surface of the electrode.

Example 17: The implantable medical device of example 16, wherein the biocompatible electrical insulator comprises parylene.

Example 18: The implantable medical device of any of example 16 or example 17, wherein the outer surface of the electrode comprises a titanium nitride (TiN) surface having an engineered surface structure, wherein the second portion of the biocompatible electrical insulator is removed without damaging the engineered surface structure.

Example 19: The implantable medical device of any one of examples 16-18, wherein the first portion of the biocompatible electrical insulator is the same as the second portion of the biocompatible electrical insulator, wherein the outer surface of the electrode comprises a titanium having a textured surface, the textured surface being formed during ablation of the biocompatible electrical insulator to remove the biocompatible electrical insulator to expose the textured surface.

Example 20: A method including: disposing a biocompatible electrical insulator on an outer surface of a housing of an implantable medical device and to cover an outer surface of an electrode, wherein the housing comprises: an electrically conductive portion defining a cavity configured to receive processing circuitry that is configured to control functioning of the implantable medical device; and a dielectric cover configured to cover the cavity and enclose the processing circuitry within the cavity, wherein the electrode is positioned on a portion of the outer surface of the dielectric cover; ablating a portion of the biocompatible electrical insulator to expose an outer surface of the electrode; and ablating the surface of the electrode to form a surface texture that increases a surface area of the surface of the electrode concurrently with ablating the portion of the biocompatible electrical insulator.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may be implemented, at least in part, in hardware, software, firmware or any combination thereof. For example, various aspects of the techniques may be implemented within one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, or other devices. The terms “processor,” “processor circuitry,” “processing circuitry,” “controller” or “control module” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as random-access memory (RAM), read-only memory (ROM), non-volatile random-access memory (NVRAM), electrically erasable programmable read-only memory (EEPROM), FLASH memory, magnetic media, optical media, or the like that is tangible. The computer-readable storage media may be referred to as non-transitory. A server, client computing device, or any other computing device may also contain a more portable removable memory type to enable easy data transfer or offline data analysis. The instructions may be executed to support one or more aspects of the functionality described in this disclosure.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various aspects of this disclosure have been described. These and other aspects are within the scope of the following claims.

The invention claimed is:

1. A method of manufacturing an implantable medical device, the method comprising:
disposing a biocompatible electrical insulator on an outer surface of a housing of the implantable medical device and to cover an outer surface of an electrode that is positioned on the outer surface of the housing;
ablating a portion of the biocompatible electrical insulator; and
removing the biocompatible electrical insulator to expose the outer surface of the electrode.

2. The method of claim 1, wherein the biocompatible electrical insulator comprises parylene.

3. The method of claim 1, wherein the outer surface of the electrode comprises a titanium nitride (TiN) surface having an engineered surface structure, wherein ablating or removing the portion of the biocompatible electrical insulator does not damage the engineered surface structure.

4. The method of claim 3, further comprising:
prior to disposing the biocompatible electrical insulator to cover the outer surface of the electrode, disposing a removable maskant on the outer surface of the electrode.

5. The method of claim 4, wherein ablating the portion of the biocompatible electrical insulator comprises scoring a first portion of the biocompatible electrical insulator adjacent to the removable maskant.

6. The method of claim 5, wherein removing the biocompatible electrical insulator comprises peeling the removable maskant and a second portion of the biocompatible electrical insulator from the outer surface of the electrode, wherein peeling the removable maskant from the outer surface of the electrode does not damage the engineered surface structure.

7. The method of claim 5, wherein the removeable maskant comprises at least one of a UV cure adhesive, a molded silicone, or a Kapton tape.

8. The method of claim 1, wherein ablating the portion of the biocompatible electrical insulator comprises the removing the biocompatible electrical insulator to expose an outer surface of the electrode.

9. The method of claim 1, wherein ablating the portion of the biocompatible electrical insulator comprises laser ablation with at least one of an ultraviolet fiber laser, a femtosecond pulsed laser, or an excimer laser.

10. The method of claim 9, wherein the outer surface of the electrode comprises a titanium surface, wherein the method further comprises texturing the titanium surface via the laser ablation.

11. The method of claim 10, wherein the texturing occurs at the same time as ablating the portion of the biocompatible electrical insulator.

12. The method of claim 1, wherein the portion of the biocompatible electrical insulator is a first portion, wherein the housing comprises:
an electrically conductive portion defining a cavity configured to receive processing circuitry, wherein the processing circuitry is configured to control functioning of the implantable medical device; and
a dielectric cover configured to cover the cavity and enclose the processing circuitry,
wherein the electrode is positioned on an outer surface of the dielectric cover and is connected to the processing circuitry, wherein the processing circuitry is configured to monitor a physiological parameter of a patient via the electrode,
wherein disposing the biocompatible electrical insulator on the outer surface of the housing comprises disposing the biocompatible electrical insulator on an outer surface of the dielectric cover,
the method further comprising:
ablating a second portion of the biocompatible electrical insulator to expose at least a portion of the outer surface of the dielectric cover.

13. The method of claim 12, wherein the portion of the outer surface of the dielectric cover corresponds to a sensor housed within the cavity, wherein the biocompatible electrical insulator reduces a transmission of an ablation energy through the dielectric cover.

14. The method of claim 12, wherein the at least the portion of the biocompatible electrical insulator is a first portion of the biocompatible electrical insulator, wherein the outer surface of the housing is a first outer surface of the housing, the method further comprising ablating a second portion of the biocompatible electrical insulator from a second portion of the housing.

15. The method of claim 14, wherein the second portion of the housing is facing a direction that is at least ninety degrees different than a direction that the first portion of the housing is facing.

16. An implantable medical device comprising:
a housing configured to house processing circuitry, wherein the processing circuitry is configured to control functioning of the implantable medical device, wherein the housing comprises:
an electrically conductive portion defining a cavity configured to receive the processing circuitry; and
a dielectric cover configured to cover the cavity and enclose the processing circuitry within the cavity;
an electrode positioned on an outer surface of the dielectric cover and opposite the processing circuitry, wherein the processing circuitry is configured to monitor a physiological parameter of a patient via the electrode; and
a biocompatible electrical insulator disposed on an outer surface of at least one of the electrically conductive portion or the dielectric cover,
wherein a first portion of the biocompatible electrical insulator is ablated, and
wherein a second portion of the biocompatible electrical insulator is removed to expose an outer surface of the electrode.

17. The implantable medical device of claim 16, wherein the biocompatible electrical insulator comprises parylene.

18. The implantable medical device of claim 16, wherein the outer surface of the electrode comprises a titanium nitride (TiN) surface having an engineered surface structure, wherein the second portion of the biocompatible electrical insulator is removed without damaging the engineered surface structure.

19. The implantable medical device of claim 16, wherein the first portion of the biocompatible electrical insulator is the same as the second portion of the biocompatible electrical insulator,
wherein the outer surface of the electrode comprises a titanium having a textured surface, the textured surface being formed during ablation of the biocompatible electrical insulator to remove the biocompatible electrical insulator to expose the textured surface.

20. A method comprising:
disposing a biocompatible electrical insulator on an outer surface of a housing of an implantable medical device and to cover an outer surface of an electrode, wherein the housing comprises:
an electrically conductive portion defining a cavity configured to receive processing circuitry that is configured to control functioning of the implantable medical device; and
a dielectric cover configured to cover the cavity and enclose the processing circuitry within the cavity, wherein the electrode is positioned on a portion of the outer surface of the dielectric cover;
ablating a portion of the biocompatible electrical insulator to expose an outer surface of the electrode; and
ablating the surface of the electrode to form a surface texture that increases a surface area of the surface of the electrode concurrently with ablating the portion of the biocompatible electrical insulator.
